(19) United States
(12) Patent Application Publication
(10) Pub. No.: US 2025/0255506 A1
(43) Pub. Date: Aug. 14, 2025

(54) SYSTEM AND METHOD FOR USING BLOOD FLOW MEASUREMENTS TO DIAGNOSE AND TREAT HEALTH FUNCTIONS

(71) Applicant: Guy P. Curtis, San Diego, CA (US)

(72) Inventor: Guy P. Curtis, San Diego, CA (US)

(21) Appl. No.: 18/913,138

(22) Filed: Oct. 11, 2024

Related U.S. Application Data
(63) Continuation of application No. 18/438,440, filed on Feb. 10, 2024.

(51) Int. Cl. A61B 5/0295; A61B 5/00; A61B 5/0205; A61B 5/021; A61B 5/024; A61B 5/1455; A61B 7/04

(52) U.S. Cl. CPC A61B 5/0295; A61B 5/0205; A61B 5/7246; A61B 5/7278; A61B 5/021; A61B 5/024; A61B 5/14551; A61B 7/04

(57) ABSTRACT

A system and methodology are provided for using a pulse oximeter to continuously measure a patient's blood flow over extended periods of time. For this purpose, a correlation factor is established which correlates a predetermined difference between wavelength colors detected by the pulse oximeter with blood pressure measurements taken by a sphygmomanometer. Variations of this correlation factor can then be subsequently monitored independently by the pulse oximeter and compared relative to previously established parameters for blood flow. Clinical personnel are thereby alerted when attention to the patient's health condition is required.

This continuation-in-part application claims the benefit of U.S. patent application Ser. No. 18/438,440 filed Feb. 10, 2024. The entire contents of application Ser. No. 18/438,440 are hereby incorporated by reference herein.

### FIELD OF THE INVENTION

The present invention pertains generally to systems and methods for noninvasively monitoring a patient's blood flow continuously over an extended period of time. The present invention is particularly, but not exclusively, pertinent to systems and methods which will accurately measure and monitor a patient's blood flow for the purposes of identifying and evaluating the hydrodynamic cardiac and circulatory performance of the patient's vascular system.

### BACKGROUND OF THE INVENTION

It is routine in clinical practice to measure a patient's peak systolic pressure together with his/her comparable diastolic pressure during a heartbeat. A comparison of these two measurements are thereafter typically referred to collectively as the patient's “blood pressure”. Heretofore this “blood pressure” measurement has been considered sufficient for diagnostic purposes. A blood pressure measurement, however, can also be used to trace changes between the amplitudes of a patient's diastolic and systolic pressures during a single heartbeat, to thereby generate a blood flow waveform.

Importantly, the patient's blood flow waveform provides information that is indicative of volumetric blood flow which is also an important diagnostic consideration. The question then is how can a volumetric blood flow value be accurately measured and monitored for clinical purposes. As envisioned by the present invention, oxygen saturation levels which are monitored and measured by a pulse oximeter can be directly correlated to the patient's blood flow waveform for this purpose.

In an evaluation of a patient's heart muscle function, the concept of time requires a dynamic perspective of the blood pressure waveform in a pulse-to-pulse comparison. For instance, time considerations set a patient's pulse rate. Further, a time sequence of pulsatile blood flow waveforms invites dynamic considerations of changes in the extremes of diastolic pressure and systolic pressure. Moreover, the time rate of pressure changes between these pressure extremes during each pulse duration is of diagnostic value. For purposes of this disclosure, all of the variables that are involved in defining a blood pressure waveform are hereinafter collectively referred to as “parametric measurements”.

As recognized for the present invention, an appreciation of the parametric measurements that define a patient's blood flow waveform, and how these parametric measurements change with time in consecutive waveforms of blood flow, can be analyzed in terms of changes in the volume of blood flow as evidenced by the blood flow waveform. This is so because it is the parametric measurements that effectively determine a blood flow waveform. Furthermore, parametric measurements also provide valuable insight into the patient's cardiac performance and peripheral resistance to blood flow in the arteries of the patient.

For the reasons set forth above, it is an object of the present invention to underscore the importance of evaluating various additional factors that will result from an analysis of a patient's blood flow waveform. Another object of the present invention is to evaluate these additional factors for further consideration and use in a comprehensive clinical diagnosis. Yet another object of the present invention is to emphasize the clinical benefits which result from a dynamic evaluation of consecutive blood flow waveforms that are based on parametric measurements of a blood flow waveform. Another object of the present invention is to establish a methodology for using a pulse oximeter to noninvasively and continuously monitor volumetric blood flow, to assess the hemodynamic cardiac and circulatory performance of a patient's vasculature. Still another object of the present invention is to provide a blood flow monitor capable of performing the above-cited objects which is easy to use, is simple to manufacture, and is comparatively cost effective.

### SUMMARY OF THE INVENTION

A blood pressure monitor in accordance with the present invention collects parametric measurements from a patient's blood pressure waveform that can be used to assess and evaluate a patient's health condition. Importantly, these parametric measurements are taken from blood pressure values that essentially define a blood flow waveform from a patient. These measurements are then compared with those of both prior and subsequently measured waveforms. This comparison thus provides a basis for a more comprehensive diagnosis of a patient's health condition based on consecutively obtained blood flow pressure measurements.

Structurally, a system for measuring the blood flow of a patient in accordance with the present invention includes a pulse oximeter of any type well known in the pertinent art. The importance here is that it is well known a pulse oximeter will trace changes in blood pressure during a patient's heartbeat. From such a trace the following parametric measurements can be obtained which are of specific importance. These include: 1) changes in diastolic pressure ±Δp_{d}; changes in systolic pressure, ±Δpₛ; and 3) changes in pulse time duration, ±Δtᵣ. Not only are these parametric measurements individually important, the comparisons of these parametric measurements relative to each other, statically and dynamically, are also important. For instance, the ratios of Δp_{d}/Δpₛ, Δpₛ/Δtᵣ, and Δp_{d}/Δtᵣ, as well as the cumulative values ΣΔp_{d}, ΣΔpₛ, and/or ΣΔtᵣ, may be informative insofar as pressure trends are concerned. Further, the time rate of rise from p_{d} to pₛ during tᵣ is considered relative to the vigor of the heart's contractions, and the slope of the pressure runoff from pₛ to p_{d} during tᵣ is considered indicative of the peripheral vascular resistance to blood flow. In each case, regardless of whether measurements are considered in a single pulse or in a consecutive pulse-to-pulse context, comparisons of parametric measurements clearly have diagnostic value.

As part of the system for monitoring blood flow, the present invention includes a computer system that receives audiometric signals from the pulse oximeter. Importantly, these signals essentially define the blood flow waveform in the vasculature of the patient. Also included within the computer system is a calculator which uses these parametric measurements from the blood flow waveform to calculate a value for the blood flow volume in the patient's vasculature. Specifically, calculations are made for each consecutive pulse of the patient's heart muscle function. From these calculations, a blood flow volume can be considered comparable to the value of an area bounded by the blood flow waveform and a timeline underneath the blood flow waveform. In this context, for the present invention the timeline is equal in value to the time pulse rate tᵣ of the patient's heart muscle function, e.g. the time between consecutive measurements of diastolic pressures, p_{d}.

Included in the computer system is a monitor that receives information from the calculator to evaluate changes in the parametric measurements of a blood flow wave form. Specifically, by comparing consecutive waveforms, the changes of +Δp_{d}, +Δpₛ, and ±Δtᵣ can be determined. Additionally, a video display is provided to present sequential values of the parametric measurements for use in evaluating the patient's health condition.

For an alternative embodiment of the present invention, the difference between raw red and infrared light wavelengths Δλ is measured during each heartbeat. The wavelength difference Δλ can then be correlated to the difference between the diastolic pressure p_{d} and systolic pressure pₛ for blood pressure Δp. To do this, the present invention relies on the use of a predetermined correlation factor Δλ/Δp. Specifically, the correlation factor correlates oxygen saturation levels of different color wavelength Δλ at the beginning and at the end of each heart beat with blood pressure measurements Δp taken by a blood pressure measuring instrument such as a sphygmomanometer. As appreciated by the present invention, once established, the correlation factor Δλ/Δp is useful to dynamically monitor blood flow.

In detail, the pulse oximeter simultaneously measures oxygen saturation levels based on color frequency differences between wavelengths in both the raw red visible spectrum λᵣᵣ and in the infrared invisible spectrum Air. In accordance with the present invention, the difference between these wavelengths, Δλ=λᵢᵣ−λᵣᵣ, is then directly correlated with a previously determined blood pressure measurement Δp=pₛ−p_{d}. For this correlation, the value difference between light wavelengths Δλ in a sequence of blood pulses can be considered constant in the correlation factor Δλ/Δp. Preferably, Δλ is determined when a patient is inactive and at rest. Similarly, the value difference between blood pressure measurements Δp is also to be constant, and is preferably determined while the patient is at rest. An important consideration here is that, although Δλ and Δp are considered constant, the predetermined wavelength values λᵢᵣ and λᵣᵣ as well as the pressure values pₛ and p_{d} are variable.

Although individual wavelength values λᵢᵣ and λᵣᵣ, and individual pressure values pₛ and p_{d} are variable, the static nature of the common correlation factor Δλ/Δp allows them to be considered collectively with each other. For example, an alarm can be activated whenever the value of a single individual wavelength value, e.g. λᵣᵣ, differs ±from a predetermined value within a predetermined timeframe. In this example, along with a detected change in λᵣᵣ, the other variables, λᵢᵣ, pₛ and p_{d} will also change with the correlation factor Δλ/Δp, because Δλ and Δp are constant.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a system for monitoring blood flow in accordance with the present invention, with the system shown operationally connected to a patient;

FIG. 2 is a block diagram of the operative components of the system showing operational interconnections for components of the present invention;

FIG. 3 is a graph showing the pressure variations of an aortic pulse during a heartbeat of the heart muscle function;

FIG. 4 is a depiction of the essential parametric measurements used for describing a pulsed blood flow volume;

FIG. 5 is a line graph showing variations of parametric measurements in a consecutive sequence of pulsed blood flow volumes in the context of a dynamic perspective of blood flow waveforms;

FIG. 6A is a portion of wavelength colors detected by a pulse oximeter which includes raw red light having a wavelength λᵣᵣ in the visible light spectrum, and infrared light having a wavelength λᵢᵣ in the invisible light spectrum;

FIG. 6B is a line graph showing the relationship between diastolic and systolic pressures in a patient's blood pressure; and

FIG. 7 is a graphical presentation of the relationship between changes in blood flow ΔF relative to pressure changes Δp.

### DESCRIPTION OF THE PREFERRED EMBODIMENTS

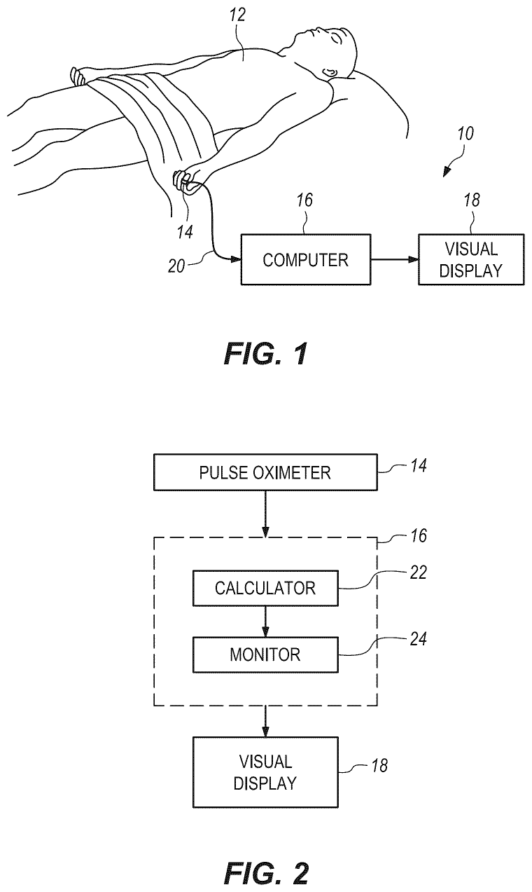

Referring initially to FIG. 1, a system for monitoring blood flow is generally designated 10. In FIG. 1 the system 10 is shown connected to a patient 12 for the purpose of measuring blood flow characteristics of the patient 12. Further, shown included in the system 10 is a pulse oximeter 14, a computer 16 and a visual display 18. In combination with other components of system 10, the pulse oximeter 14 is shown non-invasively positioned against the patient 12 to receive audiometric signals from the vasculature of the patient 12. Although the pulse oximeter 14 is shown positioned on a finger of the patient 12 in FIG. 1, the present invention envisions that the pulse oximeter 14 may be positioned on the patient 12 wherever positioning is clinically convenient. In any case, system 10 is intended to be electronically engaged with the patient 12 via a connector 20.

In FIG. 2, the computer 16 is shown to include a calculator 22 and a monitor 24. Specifically, calculator 22 is used to receive audiometric signals from the vasculature of patient 12. With these signals values are calculated based blood flow volume characteristics in the vasculature of patient 12. This is done consecutively for each pulse of the patient's heart muscle function. The monitor 24 then evaluates changes in parametric measurements of the blood flow volume as an indicator of the health condition of the patient 12. Results from this evaluation are subsequently transferred to visual display 18 for a presentation of values from the parametric measurements of the blood volume flow are provided. Clinical personnel are thereby provided with the necessary information required to accurately assess a patient's health condition.

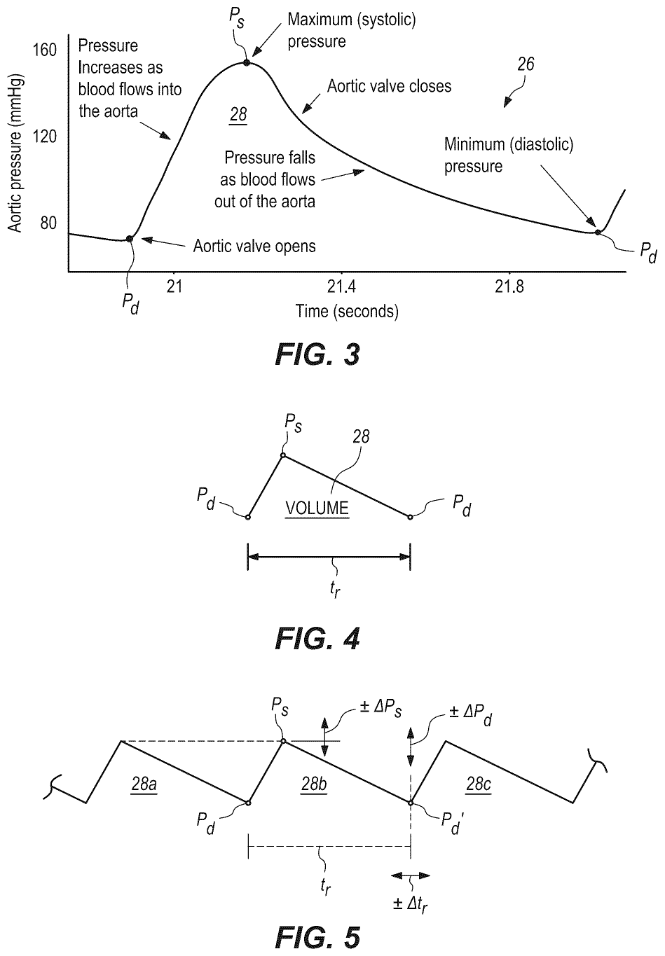

A graph 26 for a generic aortic pulse 28 is shown in FIG. 3. with annotations which illustrate and describe the time variations of aortic activities during the pulse 28. Notably, in FIG. 3 graph 26 indicates that an aortic pulse 28 can be evaluated as pressure changes in a series of connected time segments. More specifically, as shown in FIG. 4, there is a first segment in an aortic pulse 28 that occurs during a pressure rise from a diastolic pressure, p_{d}, to a systolic pressure pₛ. This first segment is then immediately followed by a second segment that occurs as the pressure falls from the systolic pressure pₛ to a diastolic pressure pa. Δt that point, another pulse 28 begins. As shown in FIG. 4, both the first and second segments of an aortic pulse 28 will occur within the pulse duration time of t_{d}.

Further, in FIG. 4 it is to be appreciated that parametric measurements from the pulse oximeter 14 can be taken to define the boundary for a blood flow volume in the vasculature of a patient 12. Specifically, for each pulse 28, the parametric measurements of diastolic pressure, p_{d}, systolic pressure pₛ, and pulse time duration tᵣ together provide reasonable values for approximating blood flow volume in the heart muscle function of patient 12 (compare FIGS. 3 and 4). It is important here to recognize that for diagnostic purposes, the dynamic values of individual parametric measurements and their variations over time alone provide valuable health information, aside from the actual blood flow volume per se. Note here also that values for the variables p_{d}, pₛ and tᵣ may vary individually or collectively from pulse to pulse.

As a technical summary for an operation of system 10 of the present invention, FIG. 5 shows a continuous sequence of pulses 28a-c which are provided for the purpose of illustrating variations in the parametric measurements being monitored. As noted above, the individual variables of diastolic pressure, p_{d}, systolic pressure, pₛ, and pulse duration, tᵣ, can be determined separately for each pulse 28 in the heart function of patient 12. It has also been noted above that each of these parametric measurements can change individually, e.g. from pulse 28a to pulse 28b, et seq. With specific reference to the pulse 28b, note that in comparison with the previous pulse 28a, it is possible that a change of systolic pressure equal to ±Δpₛ may have occurred. Further, it is also noted that during the pulse 28b, the diastolic pressure p_{d} at the beginning of the pulse 28b may change to p_{d}′ at the end of the pulse 28b. Thus, there is a change in pressure equal to ±Δp_{d} during the pulse 28b. It can also happen that the time duration tᵣ will change during between consecutive pulses 28, with an increase or decrease equal to ±Δtᵣ. Accordingly, there are many variations in parametric measurements that may have pertinent information for a further analysis of a health condition.

Depending on which aspect of a blood flow waveform is of interest, only certain variables may be important. In the specific case for the present invention, where a volumetric blood flow is of interest and is to be monitored by a pulse oximeter, the important variables are blood color wavelengths, λ, and blood pressure, p.

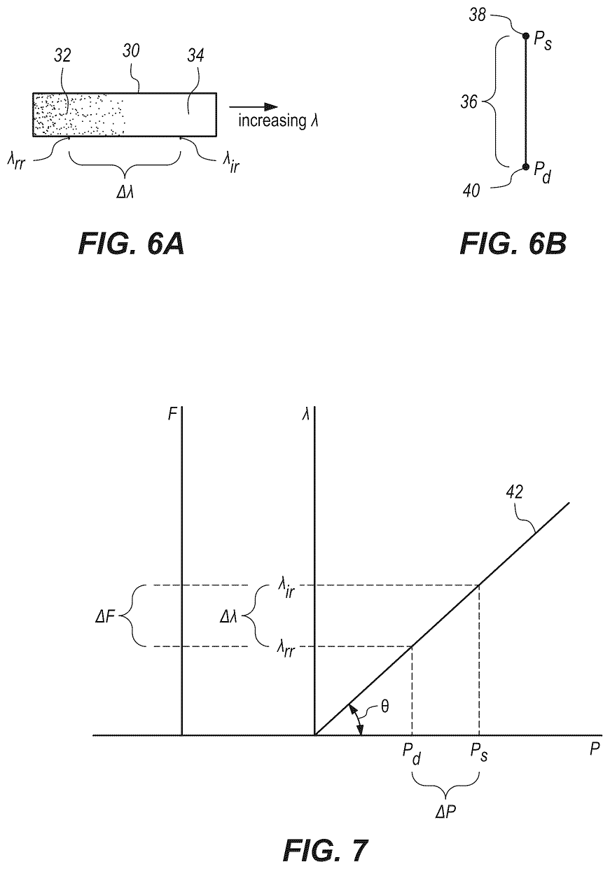

In FIG. 6A, a waveform portion 30 of a blood flow waveform is shown. Within this waveform portion 30 there is a first segment 32 which includes a wavelength λᵣᵣ of raw red color value which is in the visible light spectrum. In a second segment 34 there is another wavelength λᵢᵣ of infrared color value that is in the invisible light spectrum. For the present invention, both λᵣᵣ and λᵢᵣ are individually measured by the pulse oximeter 14 relative to a patient's heartbeat. As noted above, however, the wavelength difference between these color segments, i.e. Δλ=λᵢᵣ−λᵣᵣ, is considered separately as a static constant for successive blood pulses.

In FIG. 6B, a blood pressure measurement Δp 36 is shown which is the difference between a systolic pressure pₛ 38 and a diastolic pressure p_{d} 40. Like Δλ which is considered as a constant, the blood pressure measurement Δp=pₛ−p_{d} is also considered constant. Together, Δλ and Δp are considered together in the correlation factor 42 Δλ/Δp.

A correlation factor 42 is provided in FIG. 7 to show how a correlation factor Δλ/Δp compares its components Δλ and Δp, and how changes in blood flow ΔF are affected by changes in pressure Δp in accordance with the correlation factor Δλ/Δp. Graphically, this interaction is shown to be mathematically expressed as an inclination angle θ, where tan θ=Δλ/Δp. In any event, the correlation factor Δλ/Δp provides a reference which can be monitored to cause an alarm whenever variations in Δλ and Δp so warrant. As shown in FIG. 7, both changes in Δλ and the value of blood flow change, ΔF, are considered functions of only blood pressure differences Δp, between systolic pressure pₛ and diastolic pressure pa. As noted above, the variable ΔF is useful for measures of thermodynamic cardiac and circulatory performance.

In summary, the pulse oximeter 14 measures oxygen saturation levels based on a preselected difference in color frequency between raw red wavelength λᵣᵣ in the visible spectrum, and a wavelength λᵢᵣ the infrared invisible spectrum. In accordance with the present invention, the difference between these wavelengths, Δλ=λᵢᵣ−λᵣᵣ is directly correlated with a previously determined blood pressure measurement Δp=pₛ−p_{d}. Moreover, the difference between light wavelengths Δλ and the difference between pressure measurements Δp in a sequence of blood pulses are both considered constant in the correlation factor Δλ/Δp. Further, it is recognized that when a patient is active and is not at rest, although λᵢᵣ and λᵢᵣ are considered relatively constant, individual wavelengths and individual blood pressure measurements Δp can differ considerably.

As noted above, although individual wavelength values λᵢᵣ and λᵣᵣ, and individual pressure values pₛ and p_{d} are variable, the static nature of the common correlation factor Δλ/Δp allows them to be considered directly with each other. For example, an alarm can be activated whenever the value of a single individual wavelength value, e.g. infrared λᵢᵣ, differs ± from a predetermined value within a predetermined timeframe. Moreover, in this example, along with a detected change in λᵣᵣ, the other variables, λᵢᵣ, pₛ and p_{d} will also change with the correlation factor Δλ/Δp, because Δλ and Δp are constant. In any event, the correlation factor Δλ/Δp which can be continuously recorded and checked by a monitor and alarmed when practicable.

While the system and method for measuring blood flow in a patient as herein shown and disclosed in detail are fully capable of obtaining the objects and providing the advantages herein before stated, it is to be understood that they are merely illustrative of the presently preferred embodiments of the invention and that no limitations are intended in the details of construction or design herein shown other than as described in the appended claims.

What is claimed is:

1. A system for monitoring a patient's blood flow which comprises:
a pulse oximeter for measuring differences in the red-infrared waveform from the blood of a patient to identify a wavelength difference Δλ between red and infrared during a heat muscle function;
a calculator for comparing the wavelength difference Δλ with a blood pressure measurement Δp between diastolic and systolic pressures from the patient, to create a correlation factor Δλ/Δp indicative of blood flow in a steady state condition when the patient is at rest; and
a monitor for evaluating changes in Δp, based on the correlation factor Δλ/Δp, as an indication of a change in blood flow.

2. The system of claim 1 wherein Δλ of the correlation factor Δλ/Δp is based on a difference between a raw red color value λᵣᵣ and an infrared color value λᵢᵣ, i.e. Δλ=λᵢᵣ−λᵣᵣ, and wherein Δλ is measured by the pulse oximeter, and wherein Δp is established by an extracorporeal device which measures the systolic and diastolic pressures.

3. The system of claim 2 wherein the extracorporeal device is selected from the group consisting of a sphygmomanometer and a micromanometer.

4. The system of claim 1 wherein the steady state condition pertains to any posture of the patient while the patient is immobile.

5. The system of claim 1 wherein the red-infrared waveform comprises:
a first waveform segment from raw red light having a wavelength λᵣᵣ in the visible light spectrum; and
a second waveform segment from infrared light having a wavelength λᵢᵣ in the invisible light spectrum, wherein raw red λᵣᵣ is less than infrared λᵢᵣ.

6. The system of claim 1 wherein, based on the correlation factor Δλ/Δp, changes in Δp are deemed to be the cause of a change in blood flow.

7. The system of claim 6 further comprising an alarm feature when the value of the correlation factor Δλ/Δp differs more or less by a predetermined value within a predetermined timeframe.

8. The system of claim 7 wherein the alarm is activated whenever the correlation factor Δλ/Δp differs ±0.2 within a five minute timeframe, and whenever the correlation factor Δλ/Δp differs ±0.05 within a 30 second timeframe.

9. The system of claim 1 wherein blood flow measurements are used in hemodynamic cardiac and circulatory performance evaluations of a patient.

10. A system for monitoring a patient's blood flow which comprises:
a means for identifying a wavelength change Δλ between a raw red wavelength λᵣᵣ in a visible light spectrum and an infrared wavelength λᵢᵣ in an invisible light spectrum, wherein both are measured from the blood of a patient during a heart muscle function;
a means for comparing the wavelength change Δλ with a blood pressure measurement Δp between systolic and diastolic pressures from the patient, to create a correlation factor Δλ/Δp indicative of blood flow in a steady state condition; and
a monitor for evaluating changes in the correlation factor Δλ/Δp indicative of a change in blood flow.

11. The system of claim 10 wherein the correlation factor Δλ/Δp is a comparison wherein Δλ is measured by a pulse oximeter and Δp is established by an extracorporeal device which measures the systolic and diastolic pressures.

12. The system of claim 11 wherein the extracorporeal pressure measuring device is selected from the group consisting of a sphygmomanometer and a micromanometer.

13. The system of claim 10 wherein the steady state condition pertains to any posture of the patient while the patient is at rest.

14. The system of claim 10 wherein, based on the correlation factor Δλ/Δp, changes in Δp are deemed to be the cause of a change in blood flow.

15. The system of claim 14 further comprising an alarm feature when the value of the correlation factor Δλ/Δp differs ± from a predetermined value within a predetermined timeframe.

16. The system of claim 15 wherein the alarm is activated whenever the correlation factor Δλ/Δp differs ±0.2 within a five minute timeframe, and whenever the correlation factor Δλ/Δp differs ±0.05 within a 30 second timeframe.

17. A method for monitoring a patient's blood flow which comprises the steps of:
identifying a wavelength difference Δλ between a raw red wavelength λᵣᵣ in a visible red waveform and an infrared wavelength λᵢᵣ in an invisible infrared waveform, measured from the blood of a patient during a heat muscle function;
correlating the wavelength change Δλ=λᵢᵣ−λᵣᵣ with a blood pressure measurement Δp between systolic and diastolic pressures from the patient;
creating a correlation factor Δλ/Δp indicative of blood flow in a steady state condition when the patient is at rest; and
displaying changes in the correlation factor Δλ/Δp indicative of a change in blood flow.

18. The method of claim 17 wherein changes in Δp are deemed to be the cause of a change in blood flow.

19. The method of claim 17 further comprising the step of providing an alarm feature wherein an alarm is activated when the value of the correlation factor Δλ/Δp differs ±from a predetermined value within a predetermined timeframe.

20. The method of claim 19 wherein the alarm is activated whenever the correlation factor Δλ/Δp differs ±0.2 within a five minute timeframe, and whenever the correlation factor Δλ/Δp differs ±0.05 within a 30 second timeframe.
